(19) United States
(12) Patent Application Publication
(10) Pub. No.: US 2026/0004911 A1
(43) Pub. Date: Jan. 1, 2026

(54) Associating a designated user with a medicament delivery device

(71) Applicant: SHL Medical AG, Zug (CH)

(72) Inventors: Ola Hallström, Nacka Strand (SE); Daniel Carlsson, Nacka Strand (SE); Rasmus Renstad, Nacka Strand (SE)

(21) Appl. No.: 18/992,026

(22) PCT Filed: Jun. 28, 2023
(86) PCT No.: PCT/EP2023/067595

§ 371 Date: Jan. 7, 2025

(30) Foreign Application Priority Data
Jul. 12, 2022 (EP) 22184332.9

(51) Int. Cl. G16H 20/17; A61M 5/00; G06F 21/32

(52) U.S. Cl. CPC G16H 20/17; A61M 5/002; G06F 21/32; A61M 2205/3303

(57) ABSTRACT

A method of a receiver of associating a user with a medicament delivery device configured to communicate a medicament delivery event wirelessly with the receiver is provided. The method comprises receiving, during a preregistration phase, a first wireless signal comprising information identifying at least one medicament delivery device and biometric information identifying a user of said at least one medicament delivery device, receiving a second wireless signal identifying a medicament delivery device communicating a medicament delivery event, determining whether or not the second wireless signal comprises a device identifier received during the preregistration phase, and if so determining that the medicament delivery device identified with the received second wireless signal was used by the user identified with the biometric information for causing the medicament delivery event.

### CROSS-REFERENCE TO RELATED APPLICATIONS

The present application is a U.S. National Phase Application pursuant to 35 U.S.C. § 371 of International Application No. PCT/EP2023/067595 filed Jun. 28, 2023, which claims priority to EP Patent Application No. 22/184,332.9 filed Jul. 12, 2022. The entire disclosure contents of these applications are herewith incorporated by reference into the present application.

### TECHNICAL FIELD

The present disclosure relates to methods of a receiver of associating a designated user with a medicament delivery device configured to communicate a medicament delivery event wirelessly to the receiver, and receivers performing the methods.

### BACKGROUND

Medicament delivery devices such as for instance injectors are commonly used or delivering medicament to users suffering for instance from diabetes.

The medicament delivery device may be arranged with a tag for wireless communication of adherence data related to an injection therapy provided by the medicament delivery device, such as for example information specifying time stamp of the medicament delivery event and subsequent disposal indicating the medicament dose having been delivered. identification of dose delivery or cap removal, or other event associated with the device, batch number, type of drug or device, expiry date of medicament, etc. The tag configured to wirelessly communicate the adherence data may be a so-called radio-frequency identification (RFID), a near-field communication (NFC) tag, or even Bluetooth.

When reading the adherence data of the used medicament delivery device with an appropriate wireless receiver in for instance a home of a designated user being associated with the receiver, there may be numerous other injectors capable of wireless communication in the vicinity of the wireless receiver, used for instance by members of the same household or even neighbours.

Thus, even if the wireless receiver receives a unique medicament delivery device identifier along with the adherence data, the receiver cannot ensure that the identified medicament delivery device and the adherence data belongs to the designated user associated with the receiver, and an erroneous registration of a medical event of e.g. a family member or a neighbour may be performed by the receiver.

### SUMMARY

One objective is to solve, or at least mitigate, this problem in the art and to provide a method of a receiver of associating a designated user with a medicament delivery device configured to communicate a medicament delivery event wirelessly to the receiver.

This objective is attained in a first aspect by a method of a receiver of associating a user with a medicament delivery device configured to communicate a medicament delivery event wirelessly with the receiver. The method comprises receiving, during a preregistration phase, a first wireless signal comprising information identifying at least one medicament delivery device and biometric information identifying a user of said at least one medicament delivery device, receiving a second wireless signal identifying a medicament delivery device communicating a medicament delivery event, determining whether or not the second wireless signal comprises a device identifier received during the preregistration phase, and if so determining that the medicament delivery device identified with the received second wireless signal was used by the user identified with the biometric information for causing the medicament delivery event.

This objective is attained in a second aspect by a receiver configured to associate a user with a medicament delivery device configured to communicate a medicament delivery event wirelessly with the receiver. The receiver comprises a processing unit and a memory, said memory containing instructions executable by said processing unit, whereby the receiver is operative to receive, during a preregistration phase, a first wireless signal comprising information identifying at least one medicament delivery device and biometric information identifying a user of said at least one medicament delivery device, receive a second wireless signal identifying a medicament delivery device communicating a medicament delivery event, determine whether or not the second wireless signal comprises a device identifier received during the preregistration phase, and if so to determine that the medicament delivery device identified with the received second wireless signal was used by the user identified with the biometric information for causing the medicament delivery event.

The receiver determines whether or not a communicating a medicament delivery event has been preregistered, and if so advantageously determines that a designated user must have caused the event since the designated user was identified by means of biometric data at the time of the preregistration.

In an embodiment, the first wireless signal is received from a medicament delivery device equipped with a biometric sensor for extracting the biometric information identifying the user.

In an embodiment, the first wireless signal is received from a packaging accommodating a batch of medicament delivery devices, which packaging is equipped with a biometric sensor for extracting the biometric information identifying the user and a wireless transmitter configured to transmit the identifiers of the medicament delivery devices, and said biometric information.

In an embodiment, the biometric information is extracted from a fingerprint, iris, face or periocular feature of the user.

The objective is attained in a third aspect by a method of a receiver of associating a user with a medicament delivery device configured to communicate a medicament delivery event wirelessly with the receiver. The method comprises receiving a first wireless signal identifying a medicament delivery device communicating a medicament delivery event, receiving a second wireless signal identifying a wireless communication device, determining whether or not the wireless communication device has been preregistered for a user, and if so determining that the medicament delivery device identified with the received first wireless signal was used by the user for which the wireless communication device has been preregistered to cause the medicament delivery event.

The objective is attained in a fourth aspect by a receiver configured to associate a user with a medicament delivery device configured to communicate a medicament delivery event wirelessly with the receiver. The receiver comprises a processing unit and a memory, said memory containing instructions executable by said processing unit, whereby the receiver is operative to receive a first wireless signal identifying a medicament delivery device communicating a medicament delivery event, receive a second wireless signal identifying a wireless communication device, determine whether or not the wireless communication device has been preregistered for a user, and if so to determine that the medicament delivery device identified with the received first wireless signal was used by the user for which the wireless communication device has been preregistered to cause the medicament delivery event.

Thus, a medicament delivery event of a medicament delivery device will advantageously only be approved and registered at a receiver if a preregistered wireless communication device such as a smart phone, a tablet, a smart watch, etc., or a wireless-communication tag designated for the purpose, such as an RFID tag, is presented to the receiver when the medicament delivery event occurs.

In a fifth aspect, a computer program comprising computer-executable instructions is provided for causing a receiver of the second and fourth aspect to perform steps recited in the method according to the first aspect and third aspect, respectively, when the computer-executable instructions are executed on a processing unit included in the receiver.

In a sixth aspect, a computer program product is provided comprising a computer readable medium, the computer readable medium having the computer program according to the sixth aspect embodied thereon.

Generally, all terms used in the claims are to be interpreted according to their ordinary meaning in the technical field, unless explicitly defined otherwise herein. All references to “a/an/the element, apparatus, component, means, step, etc.” are to be interpreted openly as referring to at least one instance of the element, apparatus, component, means, step, etc., unless explicitly stated otherwise. The steps of any method disclosed herein do not have to be performed in the exact order disclosed, unless explicitly stated.

### BRIEF DESCRIPTION OF THE DRAWINGS

Aspects and embodiments are now described, by way of example, with reference to the accompanying drawings, in which:

FIG. 1 illustrates a prior art scenario;

FIG. 2 illustrates a receiver of an embodiment;

FIG. 3 shows a flowchart illustrating a method of a receiver of associating a user with a medicament delivery device configured to communicate a medicament delivery event wirelessly to the receiver according to an embodiment;

FIG. 4 illustrates a receiver of a further aspect;

FIG. 5 shows a flowchart illustrating a method of yet a further embodiment; and

FIG. 6 illustrates a receiver of a further embodiment.

### DETAILED DESCRIPTION

The aspects of the present disclosure will now be described more fully hereinafter with reference to the accompanying drawings, in which certain embodiments of the invention are shown.

These aspects may, however, be embodied in many different forms and should not be construed as limiting; rather, these embodiments are provided by way of example so that this disclosure will be thorough and complete, and to fully convey the scope of all aspects of invention to those skilled in the art. Like numbers refer to like elements throughout the description.

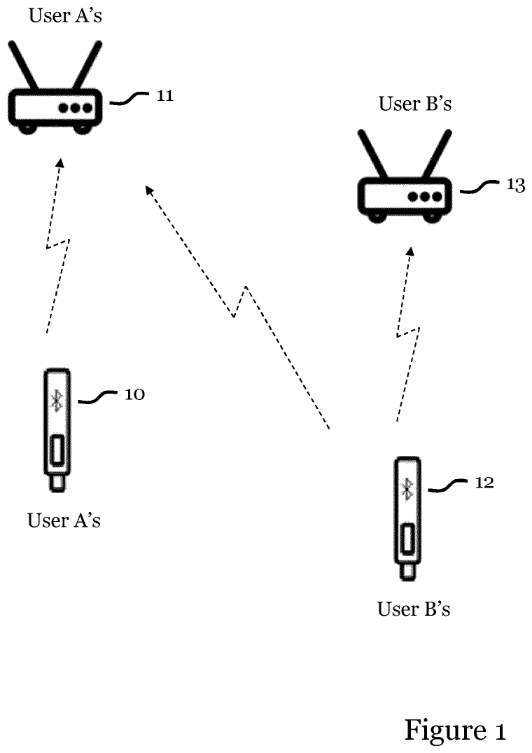

FIG. 1 illustrates a prior art scenario where upon a user utilizing a first medicament device 10, in the following exemplified in the form of an injector, for delivering medicament, the first injector 10 wirelessly transmits a signal to a first wireless receiver 11 that a medicament delivery event has occurred along with appropriate data (commonly referred to as adherence data) and an identifier for the first injector 10.

Upon receiving the signal, the first receiver 11 concludes from the identifier that the signal originates from the first device 10 and will further assume that the medicament delivery event was triggered by a designated user associated with the first receiver 11, in the following referred to as user A.

As mentioned, the wireless communication may be established using any appropriate wireless channel, such as e.g. RFID, NFC, Bluetooth, long range (LoRa) technology or ultra-wideband (UWB) radio.

However, assuming that a second injector 12 communicating with a second wireless receiver 13 is in a vicinity of the first receiver 11. The second injector 12 and second receiver 13 may be used by a household member or a neighbour depending on wireless signal range, referred to as user B being associated with the second receiver 13.

Hence, it may occur that the first receiver 11 is within the range of the second injector 12, and the first receiver 11 receiving the identifier and adherence data of the second injector 12 erroneously assumes that the medicament delivery event was triggered by user A.

As a consequence, in addition to incorrectly registering the medicament delivery event of the second injector 12 as being triggered by user A, the first receiver will further receive incorrect adherence data which really belongs to user B. As is understood, the second receiver 13 may erroneously register a medicament delivery even of user A by receiving signals from the first injector 10.

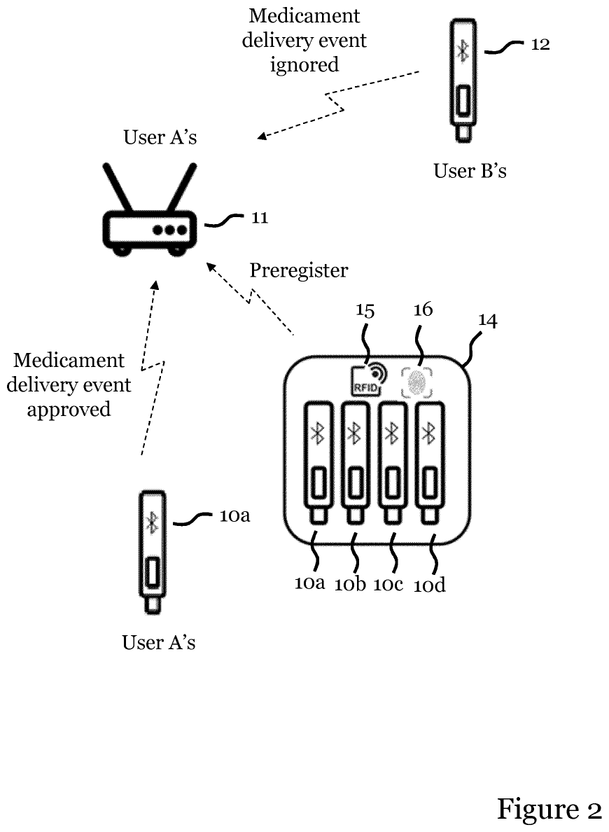

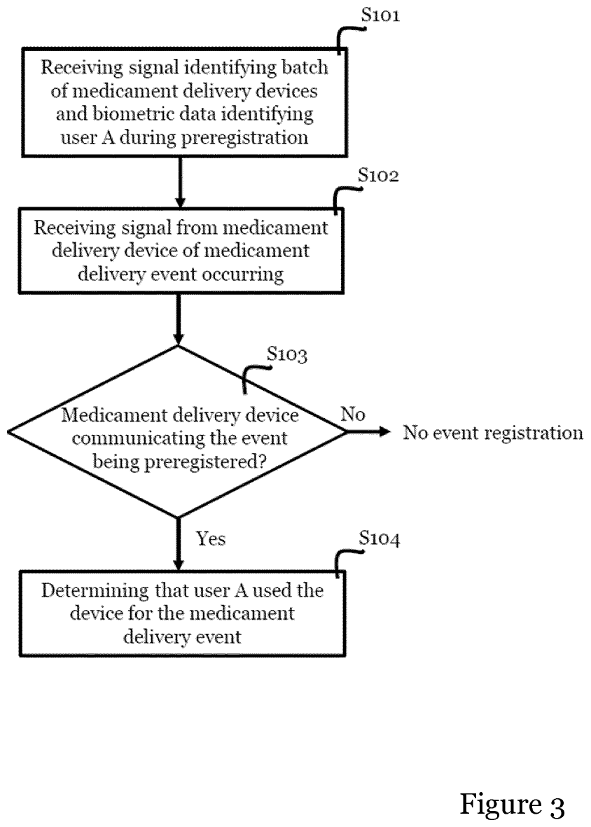

To resolve this issue, reference is made to FIG. 2 illustrating an embodiment. Reference is further made to FIG. 3 showing a flowchart illustrating a method of a receiver (i.e. first receiver 11) of associating a user (i.e. user A) with a medicament delivery device (i.e. first injector 10) configured to communicate a medicament delivery event wirelessly to the receiver.

As shown in FIG. 2, a packaging 14 accommodating a batch of injectors 10a-10d is equipped with a wireless data transmitter 15 (in this example an RFID tag) and a biometric sensor 16 in the form of a fingerprint reader.

Alternatively, an iris, face or periocular feature reader may be used, where periocular features typically are extracted from a periocular region of the user, which is the area around the eye including features like eyelashes, eyebrows, eyelids, eye shape, tear duct, skin texture, etc.

Thus, in a preregistration phase, user A registers the identifier of each injector 10a-10d in the batch along with her fingerprint with the first receiver 11. The user simply presses a finger, such as a thumb, against the fingerprint reader 16, wherein a first wireless signal comprising the identifier of the respective injector 10a-10d and fingerprint data identifying user A is transmitted in step S101 to the first receiver 11 via the RFID tag 15 of the packaging 14.

As is understood, the injectors 10a-10d may be preregistered one at a time, and it may also be envisaged that each injector 10a-10 comprises a biometric sensor. However, equipping the packaging 14 with a fingerprint sensor 16 for batch-registering is far more cost-effective and less time-consuming.

Now, upon a user causing a medicament delivery event while utilizing one of the preregistered injectors, in this case the first injector 10a, a second signal identifying the first injector 10a is transmitted from the first injector 10a and received at the first receiver 11 in step S102.

The first receiver 11 determines in step S103 whether or not the first injector 10a communicating the medicament delivery event has been preregistered, and if so advantageously determines in step S104 that user A must have caused the event since user A was identified by means of fingerprint data at the time of the preregistration.

In practice, the first receiver 11 will in step S104 register the communicated medicament delivery event (along with any adherence data) with user A.

In contrast, a second injector 12 utilized by user B has not been preregistered with the first receiver 11 and a signal received from the second injector 12 will be ignored, or at least not registered after the first receiver 11 has concluded that the identifier of the second injector 12 has not been preregistered.

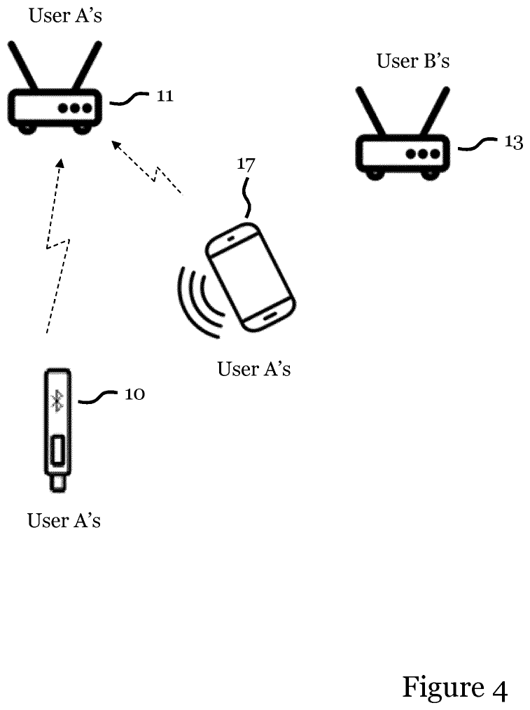

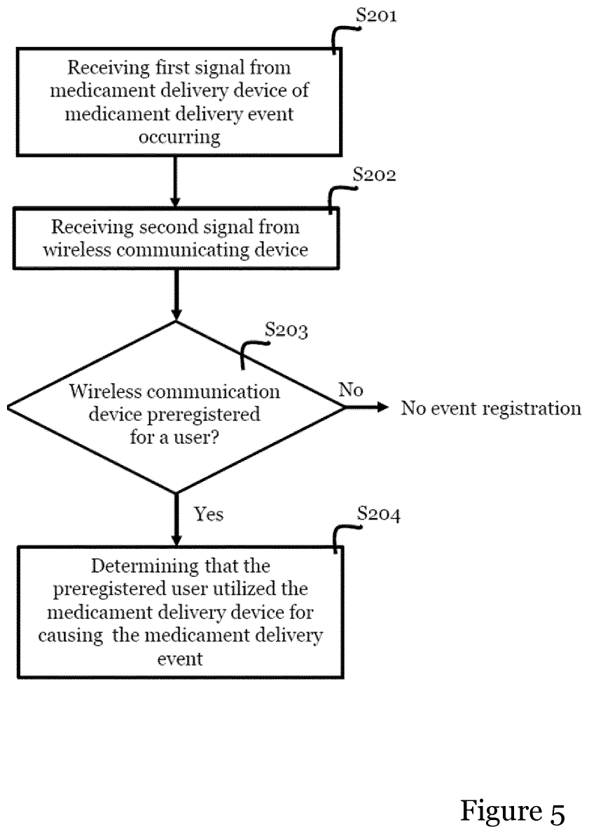

In a further aspect, with reference to FIGS. 4 and 5, a medicament delivery event of a device 10 will only be approved and registered at a receiver 11 if a preregistered wireless communication device 17 such as a smart phone, a tablet, a smart watch, etc., or a wireless-communication tag designated for the purpose, such as an RFID tag, is presented to the receiver 11 when the medicament delivery event occurs.

Hence, in a first step S201, the first wireless receiver 11 receives a first wireless signal identifying the first injector 10 and thus that a medicament delivery event has occurred.

In a second step S202, a second wireless signal identifying a wireless communication device is received, in this example the smart phone 17.

The first receiver 11 will thereafter determine in step S203 whether or not the smart phone 17 identified with the second signal has been preregistered (either directly with the first receiver 11 or via e.g. a back-end cloud server in communication with the receiver 11).

If so, the first receiver 11 concludes in step S204 that the user that was preregistered with the smart phone 17 indeed is the user which utilized the first injector 11 to cause the medicament delivery event.

Even if the second receiver 12 would receive a signal from the first injector 11 as well as from the smart phone 17, the second receiver 11 will not register the medicament delivery event of the first injector to user B, since the smart phone 17 of user A has not been preregistered with the second receiver.

Similarly, the first receiver 11 will not register a medicament delivery event of the second injector 12, since the first receiver 11 in such case would pick up a signal of a wireless communication device of user B, which has not been preregistered with the first receiver 11.

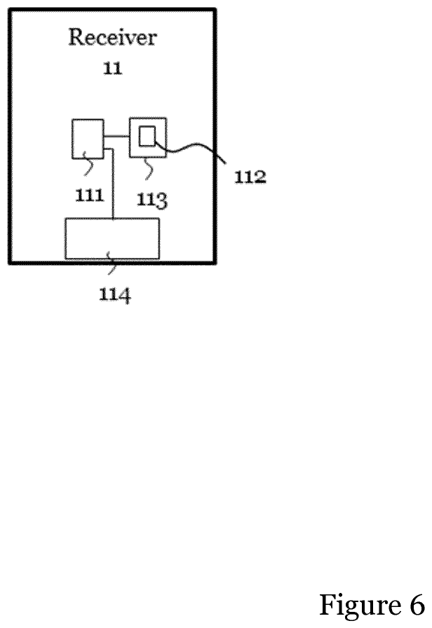

FIG. 6 illustrates a receiver 11 configured to associate a user with a medicament delivery device configured to communicate a medicament delivery event wirelessly to the receiver 11 according to an embodiment, where the steps of the method performed by the receiver 11 in practice are performed by a processing unit 111 embodied in the form of one or more microprocessors arranged to execute a computer program 112 downloaded to a storage medium 113 associated with the microprocessor, such as a Random Access Memory (RAM), a Flash memory or a hard disk drive. The processing unit 111 is arranged to cause the receiver 11 to carry out the method according to embodiments when the appropriate computer program 112 comprising computer-executable instructions is downloaded to the storage medium 113 and executed by the processing unit 111. The storage medium 113 may also be a computer program product comprising the computer program 112. Alternatively, the computer program 112 may be transferred to the storage medium 113 by means of a suitable computer program product, such as a Digital Versatile Disc (DVD) or a memory stick. As a further alternative, the computer program 112 may be downloaded to the storage medium 113 over a network. The processing unit 111 may alternatively be embodied in the form of a digital signal processor (DSP), an application specific integrated circuit (ASIC), a field-programmable gate array (FPGA), a complex programmable logic device (CPLD), etc. The receiver 11 further comprises a communication interface 114 (wired and/or wireless) over which the receiver 11 is configured to transmit and receive data using e.g. RFID, NFC, Bluetooth, LoRa technology or UWB radio as previously mentioned.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The aspects of the present disclosure have mainly been described above with reference to a few embodiments and examples thereof. However, as is readily appreciated by a person skilled in the art, other embodiments than the ones disclosed above are equally possible within the scope of the invention, as defined by the appended patent claims.

Thus, while various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1-14. (canceled)

15. A method of a receiver of associating a user with a medicament delivery device configured to communicate a medicament delivery event wirelessly with the receiver, comprising:
receiving, during a preregistration phase, a first wireless signal comprising information identifying at least one medicament delivery device and biometric information identifying a user of said at least one medicament delivery device;
receiving a second wireless signal identifying a medicament delivery device communicating a medicament delivery event;
determining whether or not the second wireless signal comprises a device identifier received during the preregistration phase; and if so
determining that the medicament delivery device identified with the received second wireless signal was used by the user identified with the biometric information for causing the medicament delivery event.

16. The method of claim 15, the first wireless signal being received from a medicament delivery device equipped with a biometric sensor for extracting the biometric information identifying the user.

17. The method of claim 15, the first wireless signal being received from a packaging accommodating a batch of medicament delivery devices, which packaging is equipped with a biometric sensor for extracting the biometric information identifying the user and a wireless transmitter configured to transmit the identifiers of the medicament delivery devices, and said biometric information.

18. The method of claim 15, the biometric information being extracted from a fingerprint, iris, face or periocular feature of the user.

19. A method of a receiver of associating a user with a medicament delivery device configured to communicate a medicament delivery event wirelessly with the receiver, comprising:
receiving a first wireless signal identifying a medicament delivery device communicating a medicament delivery event;
receiving a second wireless signal identifying a wireless communication device;
determining whether or not the wireless communication device has been preregistered for a user; and if so
determining that the medicament delivery device identified with the received first wireless signal was used by the user for which the wireless communication device has been preregistered to cause the medicament delivery event.

20. The method of claim 19, the wireless communication device being one of a smart phone, a tablet, a smart watch, and a wireless-communication tag.

21. A computer program comprising computer-executable instructions for causing a receiver to perform steps recited claim 15 when the computer-executable instructions are executed on a processing unit included in the receiver.

22. A computer program product comprising a computer readable medium, the computer readable medium having the computer program according to claim 21 embodied thereon.

23. A receiver configured to associate a user with a medicament delivery device configured to communicate a medicament delivery event wirelessly with the receiver, the receiver comprising a processing unit and a memory, said memory containing instructions executable by said processing unit, whereby the receiver is operative to:
receive, during a preregistration phase, a first wireless signal comprising information identifying at least one medicament delivery device and biometric information identifying a user of said at least one medicament delivery device;
receive a second wireless signal identifying a medicament delivery device communicating a medicament delivery event;
determine whether or not the second wireless signal comprises a device identifier received during the preregistration phase; and if so to
determine that the medicament delivery device identified with the received second wireless signal was used by the user identified with the biometric information for causing the medicament delivery event.

24. The receiver of claim 23, further being operative to receive the first wireless signal from a medicament delivery device equipped with a biometric sensor for extracting the biometric information identifying the user.

25. The receiver of claim 23, further being operative to receive the first wireless signal from a packaging accommodating a batch of medicament delivery devices, which packaging is equipped with a biometric sensor for extracting the biometric information identifying the user and a wireless transmitter configured to transmit the identifiers of the medicament delivery devices, and said biometric information.

26. The receiver of claim 23, the biometric information being extracted from a fingerprint, iris, face or periocular feature of the user.

27. A receiver configured to associate a user with a medicament delivery device configured to communicate a medicament delivery event wirelessly with the receiver, the receiver comprising a processing unit and a memory, said memory containing instructions executable by said processing unit, whereby the receiver is operative to:
receive a first wireless signal identifying a medicament delivery device communicating a medicament delivery event;
receive a second wireless signal identifying a wireless communication device;
determine whether or not the wireless communication device has been preregistered for a user; and if so to
determine that the medicament delivery device identified with the received first wireless signal was used by the user for which the wireless communication device has been preregistered to cause the medicament delivery event.

28. The receiver of claim 27, the wireless communication device being one of a smart phone, a tablet, a smart watch, and a wireless-communication tag.
